(19) United States
(12) Patent Application Publication
(10) Pub. No.: US 2008/0003609 A1
(43) Pub. Date: Jan. 3, 2008

(54) Method of detecting bladder urothelial carcinoma

(75) Inventor: Bin Yang, Solon, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/801,676

(22) Filed: May 10, 2007

Related U.S. Application Data
(60) Provisional application No. 60/799,089, filed on May 10, 2006.

(51) Int. Cl. C12Q 1/68

(52) U.S. Cl. 435/6

(57) ABSTRACT

A diagnostic method for bladder urethelial carcinoma includes obtaining an isolated nucleotide sample from a subject and detecting the promoter methylation of at least three tumor suppressor genes selected form group consisting of DAPK, RAR-beta, p14, p73, MGMT, APC, SOCS-1, BRCA-1, and FHIT.

### RELATED APPLICATION

This application claims priority from U.S. Provisional Application No. 60/799,089, filed May 10, 2006, the subject matter of which is incorporated herein by reference.

### FIELD OF THE INVENTION

The present invention relates to a method of detecting bladder urethelial carcinoma and, particularly relates to a method of detecting bladder urethelial carcinoma using DNA promoter methylation profiling.

### BACKGROUND

Bladder cancer is one of the most common neoplasms, with more than 50,000 newly diagnosed cases in the United States alone each year. Although superficial bladder transitional cell carcinoma (TCC) can be removed by transurethral resection, more than 50% recur, approximately 30% progress to invasive disease, and up to 30% of patients die from the disease. Current surveillance of patients with bladder TCC is performed by voided urine cytology followed by cytoscopy. Although high-grade flat urethelial carcinoma in situ (CIS) can be readily detected in urine by cytology, the sensitivity for cytological detection of papillary bladder TCC in urine is less than 30% due to overlapping cytomorphologic features between papillary TCC and benign/reactive processes.

DNA methylation is a mechanism for changing the base sequence of DNA without altering its coding function. DNA methylation is a heritable, reversible and epigenetic change. Yet, DNA methylation has the potential to alter gene expression, which has profound developmental and genetic consequences. The methylation reaction involves flipping a target cytosine out of an intact double helix to allow the transfer of a methyl group from S-adenosylmethionine in a cleft of the enzyme DNA (cystosine-5)-methyltransferase (Klimasauskas et al., Cell 76:357-369, 1994) to form 5-methylcytosine (5-mCyt). This enzymatic conversion is the only epigenetic modification of DNA known to exist in vertebrates and is essential for normal embryonic development (Bird, Cell 70:5-8, 1992; Laird and Jaenisch, Human Mol. Genet. 3:1487-1495, 1994; and Bestor and Jaenisch, Cell 69:915-926, 1992). The presence of 5-mCyt at CpG dinucleotides has resulted in a 5-fold depletion of this sequence in the genome during vertebrate evolution, presumably due to spontaneous deamination of 5-mCyt to T (Schoreret et al., Proc. Natl. Acad. Sci. USA 89:957-961, 1992). Those areas of the genome that do not show such suppression are referred to as “CpG islands” (Bird, Nature 321:209-213, 1986; and Gardiner-Garden et al., J. Mol. Biol. 196:261-282, 1987). These CpG island regions comprise about 1% of vertebrate genomes and also account for about 15% of the total number of CpG dinucleotides (Bird, Infra.). CpG islands are typically between 0.2 to about 1 kb in length and are located upstream of many housekeeping and tissue-specific genes, but may also extend into gene coding regions. Therefore, it is the methylation of cytosine residues within CpG islands in somatic tissues, which is believed to affect gene function by altering transcription (Cedar, Cell 53:3-4, 1988).

Methylation of cytosine residues contained within CpG islands of certain genes has been inversely correlated with gene activity. This could lead to decreased gene expression by a variety of mechanisms including, for example, disruption of local chromatin structure, inhibition of transcription factor-DNA binding, or by recruitment of proteins which interact specifically with methylated sequences indirectly preventing transcription factor binding. In other words, there are several theories as to how methylation affects mRNA transcription and gene expression, but the exact mechanism of action is not well understood. Some studies have demonstrated an inverse correlation between methylation of CpG islands and gene expression, however, most CpG islands on autosomal genes remain unmethylated in the germline and methylation of these islands is usually independent of gene expression. Tissue-specific genes are usually unmethylated and the receptive target organs but are methylated in the germline and in non-expressing adult tissues. CpG islands of constitutively-expressed housekeeping genes are normally unmethylated in the germline and in somatic tissues.

Abnormal methylation of CpG islands associated with tumor suppressor genes may also cause decreased gene expression. Increased methylation of such regions may lead to progressive reduction of normal gene expression resulting in the selection of a population of cells having a selective growth advantage (i.e., a malignancy).

It is considered that altered DNA methylation patterns, particularly methylation of cytosine residues, cause genome instability and are mutagenic. This, presumably, has led to an 80% suppression of a CpG methyl acceptor site in eukaryotic organisms, which methylate their genomes. Cytosine methylation further contributes to generation of polymorphism and germ-line mutations and to transition mutations that inactivate tumor-suppressor genes (Jones, Cancer Res. 56:2463-2467, 1996). Methylation is also required for embryonic development of mammals (Bestor and Jaenisch, Cell 69:915-926, 1992). It appears that that the methylation of CpG-rich promoter regions may be blocking transcriptional activity. Therefore, there is a probability that alterations of methylation are an important epigenetic criteria and can play a role in carcinogenesis in general due to its function of regulating gene expression. Ushijima et al. (Proc. Natl. Acad. Sci. USA 94:2284-2289, 1997) characterized and cloned DNA fragments that show methylation changes during murine hepatocarcinogenesis. Data from a group of studies of altered methylation sites in cancer cells show that it is not simply the overall levels of DNA methylation that are altered in cancer, but changes in the distribution of methyl groups.

These studies suggest that methylation, at CpG-rich sequences known as CpG islands, provide an alternative pathway for the inactivation of tumor suppressors, despite the fact that the supporting studies have analyzed only a few restriction enzyme sites without much knowledge as to their relevance to gene control. These reports suggest that methylation of CpG oligonucleotides in the promoters of tumor suppressor genes can lead to their inactivation. Other studies provide data that suggest that alterations in the normal methylation process are associated with genomic instability (Lengauer et al. Proc. Natl. Acad. Sci. USA 94:2545-2550, 1997). Such abnormal epigenetic changes may be found in many types of cancer and can, therefore, serve as potential markets for oncogenic transformation, provided that there is a reliable means for rapidly determining such epigenetic changes. The present invention was made to provide such a universal means for determining abnormal epigenetic changes and address this need in the art.

### SUMMARY OF THE INVENTION

The present invention relates to a diagnostic method for bladder urethelial carcinoma. In the method, an isolated nucleotide sample is obtained from a subject. The promoter methylation of tumor suppressor genes selected form group consisting of DAPK, RAR-beta, p14, p73, MGMT, APC, SOCS-1, BRCA-1, and FHIT is then determined. Detection of promoter methylation of three tumor suppressor genes is indicative of bladder urethelial carcinoma.

In an aspect of the invention the isolated nucleic sample can comprise isolated genomic DNA. The isolated genomic DNA can be obtained from at least one of tissue or biological fluid of the subject, for example, voided or instrumented urine samples. The detection of promoter methylation of the at least three tumor suppressor genes can distinguish bladder transition cell carcinoma from benign or malignant urethelial cells. The methylation detection of the at three tumor suppressor genes can be performed by multiplex methylation specific PCR.

### BRIEF DESCRIPTION OF THE DRAWING

FIG. 1 illustrates a flow diagram illustrating a diagnostic method in accordance with an aspect of the invention.

### DEFINITIONS

To facilitate an understanding of the present invention, a number of terms and phrases are defined below:

As used herein, the term “subject suspected of having cancer” refers to a subject that presents one or more symptoms indicative of a cancer. A subject suspected of having cancer may also have on or more risk factors. A subject suspected of having cancer has generally not been tested for cancer. However, a “subject suspected of having cancer” encompasses an individual who has received an initial diagnosis (e.g., a CT scan showing a mass) but for whom the sub-type or stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, genetic predisposition, environmental expose, preexisting non cancer diseases, and lifestyle.

As used herein, the term “stage of cancer” refers to a numerical measurement of the level of advancement of a cancer. Criteria used to determine the stage of a cancer include, but are not limited to, the size of the tumor, whether the tumor has spread to other parts of the body and where the cancer has spread (e.g., within the same organ or region of the body or to another organ).

As used herein, the term “providing a prognosis” refers to providing information regarding the impact of the presence of cancer (e.g., as determined by the diagnostic methods of the present invention) on a subject's future health (e.g., expected morbidity or mortality).

As used herein, the term “subject diagnosed with a cancer” refers to a subject having cancerous cells. The cancer may be diagnosed using any suitable method, including but not limited to, the diagnostic methods of the present invention.

As used herein, the term “detecting the presence or absence of DNA methylation” refers to the detection of DNA methylation in the promoter region of one or more genes (e.g., cancer markers of the present invention) of a genomic DNA sample. The detecting may be carried out using any suitable method, including, but not limited to, those disclosed herein.

As used herein, the term “determining a chance of disease-free survival” refers to the determining the likelihood of a subject diagnosed with cancer surviving without the recurrence of cancer (e.g., metastatic cancer). In some embodiments, determining a chance of disease free survival comprises determining the DNA methylation pattern of the subject's genomic DNA.

As used herein, the term “determining the risk of developing metastatic disease” refers to likelihood of a subject diagnosed with cancer developing metastatic cancer. In some embodiments, determining the risk of developing metastatic disease comprises determining the DNA methylation pattern of the subject's genomic DNA.

As used herein, the term “monitoring disease progression in said subject” refers to the monitoring of any aspect of disease progression, including, but not limited to, the spread of cancer, the metastasis of cancer, and the development of a pre-cancerous lesion into cancer. In some embodiments, monitoring disease progression comprises determining the DNA methylation pattern of the subject's genomic DNA.

As used herein, the term “methylation profile” refers to a presentation of methylation status of one or more cancer marker genes in a subject's genomic DNA. In some embodiments, the methylation profile is compared to a standard methylation profile comprising a methylation profile from a known type of sample (e.g., cancerous or non-cancerous samples or samples from different stages of cancer). In some embodiments, methylation profiles are generated using the methods of the present invention. The profile may be presented as a graphical representation (e.g., on paper or on a computer screen), a physical representation (e.g., a gel or array) or a digital representation stored in computer memory.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragment are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

DNA molecules are said to have “5′ ends” and “3′ ends” because mononucleotides are reacted to make oligonucleotides or polynucleotides in a manner such that the 5′ phosphate of one mononucleotide pentose ring is attached to the 3′ oxygen of its neighbor in one direction via a phosphodiester linkage. Therefore, an end of an oligonucleotide or polynucleotide is referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide or polynucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements that direct transcription of a linked gene are generally located 5′ or upstream of the coding region. However, enhancer elements can exert their effect even when located 3′ of the promoter element or the coding region. Transcription termination and polyadenylation signals are located 3′ or downstream of the coding region.

As used herein, the terms “an oligonucleotide having a nucleotide sequence encoding a gene” and “polynucleotide having a nucleotide sequence encoding a gene,” means a nucleic acid sequence comprising the coding region of a gene or in other words the nucleic acid sequence that encodes a gene product. The coding region may be present in a cDNA, genomic DNA or RNA form. When present in a DNA form, the oligonucleotide or polynucleotide may be single-stranded (i.e., the sense strand) or double-stranded. Suitable control elements such as enhancers/promoters, splice junctions, polyadenylation signals, etc. may be placed in close proximity to the coding region of the gene if needed to permit proper initiation of transcription and/or correct processing of the primary RNA transcript. Alternatively, the coding region utilized in the expression vectors of the present invention may contain endogenous enhancers/promoters, splice junctions, intervening sequences, polyadenylation signals, etc. or a combination of both endogenous and exogenous control elements.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc. (defined infra).

Transcriptional control signals in eukaryotes comprise “promoter” and “enhancer” elements. Promoters and enhancers consist of short arrays of DNA sequences that interact specifically with cellular proteins involved in transcription (T. Maniatis et al., Science 236:1237 [1987]). Promoter and enhancer elements have been isolated from a variety of eukaryotic sources including genes in yeast, insect and mammalian cells, and viruses (analogous control elements, i.e., promoters, are also found in prokaryote). The selection of a particular promoter and enhancer depends on what cell type is to be used to express the protein of interest. Some eukaryotic promoters and enhancers have a broad host range while others are functional in a limited subset of cell types (for review see, Voss et al., Trends Biochem. Sci., 11:287 [1986]; and T. Maniatis et al., supra). For example, the SV40 early gene enhancer is very active in a wide variety of cell types from many mammalian species and has been widely used for the expression of proteins in mammalian cells (Dijkema et al., EMBO J. 4:761 [1985]). Two other examples of promoter/enhancer elements active in a broad range of mammalian cell types are those from the human elongation factor 1.alpha. gene (Uetsuki et al., J. Biol. Chem., 264:5791 [1989]; Kim et al., Gene 91:217 [1990]; and Mizushima and Nagata, Nuc. Acids. Res., 18:5322 [1990]) and the long terminal repeats of the Rous sarcoma virus (Gorman et al., Proc. Natl, Acad. Sci. USA 79:6777 [1982]) and the human cytomegalovirus (Boshart et al., Cell 41:521 [1985]). Some promoter elements serve to direct gene expression in a tissue-specific manner.

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats of retroviruses contain both promoter and enhancer functions. The enhancer/promoter may be “endogenous” or “exogenous” or “heterologous.” An “endogenous” enhancer/promoter is one that is naturally linked with a given gene in the genome. An “exogenous” or “heterologous” enhancer/promoter is one that is placed in juxtaposition to a gene by means of genetic manipulation (i.e., molecular biological techniques such as cloning and recombination) such that transcription of that gene is directed by the linked enhancer/promoter.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Q_{β} replicase, MDV-1 RNA is the specific template for the replicase (Kacian et al., Proc. Natl. Acad. Sci. USA 69:3038 [1972]). Other nucleic acid will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides or polynucleotides, where there is a mismatch between the oligonucleotide or polynucleotide substrate and the template at the ligation junction (Wu and Wallace, Genomics 4:560 [1989]). Finally, Taq and Pfu polymerases, by virtue of their ability to function at high temperature, are found to display high specificity for the sequences bounded and thus defined by the primers; the high temperature results in thermodynamic conditions that favor primer hybridization with the target sequences and not hybridization with non-target sequences (H. A. Erlich (ed.), PCR Technology, Stockton Press [1989]).

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

As used herein, the term “polymerase chain reaction” (“PCR”) refers to the method of K. B. Mullis U.S. Pat. Nos. 4,683,195 4,683,202, and 4,965,188, hereby incorporated by reference, which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide or polynucleotide sequence can be amplified with the appropriate set of primer molecules. In particular, the amplified segments created by the PCR process are, themselves, efficient templates for subsequent PCR amplifications.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids, such as DNA and RNA, found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

### DETAILED DESCRIPTION

The present invention is based upon the discovery that the promoter methylation profile of a panel of tumor suppressor genes (TSG) can be used to detect bladder transitional cell carcinoma and particularly distinguish both flat and papillary bladder transitional cell carcinoma from benign or reactive urethelial cells. This is the first time that promoter methylation profiling of a panel of particular TSGs, such DAPK, RAR-beta, p14, p73, MGMT, APC, SOCS-1, BRCA-1, and FHIT has been associated with bladder urethelial carcinoma.

It has been determined that concurrent methylation of the promoters of multiple genes of a panel of tumor suppressor genes is diagnostic for the presence or potential development of bladder urethelial cell carcinoma in subjects. More particularly, the methylation of certain nucleotides localized in CpG islands of particular promoters of tumor suppressor genes has been shown to affect the expression of tumor suppressor genes associated with the CpG islands; typically such methylated genes have reduced or abolished expression, primarily due to down-regulated transcription. Although single promoter methylation of a tumor suppressor genes can be seen in benign/reactive urethelial cells, concurrent promoter methylation of CpG islands of at least three tumor suppressor genes from a panel of tumor suppressor genes, selected from the group consisting of DAPK, RAR-beta, p14, p73, MGMT, APC, SOCS-1, BRCA-1, and FHIT is only seen in papillary transition cell carcinoma (TCC) and flat urethelial cell carcinoma in situ (CIS). This concurrent promoter methylation of CpG islands of at least three tumor suppressor genes selected from the group consisting of DAPK, RAR-beta, p14, p73, MGMT, APC, SOCS-1, BRCA-1, and FHIT therefore allows one to readily distinguish papillary transition cell carcinoma (TCC) and flat urethelial cell carcinoma in situ (CIS) from benign/reactive urethelial cells.

Using concurrent promoter methylation profiling of multiple tumor suppressor genes, such as tumor suppressor genes selected from the group consisting of DAPK, RAR-beta, p14, p73, MGMT, APC, SOCS-1, BRCA-1, and FHIT, the sensitivity of in detecting bladder transitional cell carcinoma in urine is about 87% with a specificity approaching about 100%. In comparison, the sensitivity for cytological detection of low-grade papillary transitional cell carcinoma, high-grade papillary transitional cell carcinoma, and flat urethelial cell carcinoma in situ (CIS) are about 13%, about 53%, and about 85% respectively. Using a recently developed PCR-based technique called mutiplex methylation specific PCR, aberrantly methylated nucleic acids in biological samples (e.g., voided or instrumented urine samples) from individuals with bladder urethelial carcinoma can be identified.

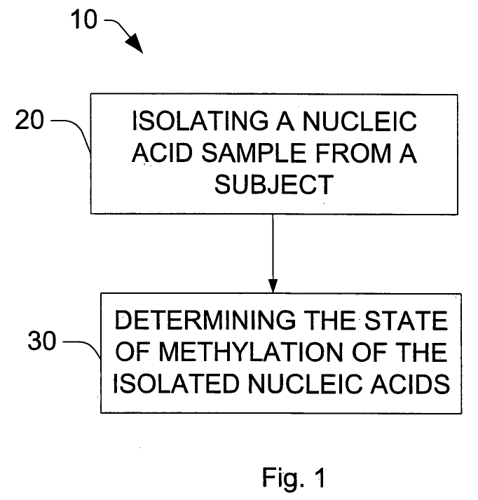

In a first embodiment, the invention provides a method of diagnosing a bladder urethelial carcinoma in a subject. In the method 10 as shown schematically in FIG. 1, at 20, at least three or more nucleic acids corresponding to promoters of TSG selected from the group consisting of DAPK, RAR-beta, p14, p73, MGMT, APC, SOCS-1, BRCA-1, and FHIT are isolated from the subject suspected of having bladder urethelial carcinoma. At 30, the state of methylation of the nucleic acids corresponding to the promoters is determined. The state of methylation of the nucleic acids from the subject suspected of having as compared with the state of methylation of similar isolated nucleic acids from a subject not having the bladder urethelial carcinoma is indicative of a bladder urethelial cell carcinoma in the subject.

The method includes detecting the methylation profiles of at three or more nucleic acids corresponding to promoters of TSGs isolated from the subject selected from the group consisting of DAPK, RAR-beta, p14, p73, MGMT, APC, SOCS-1, BRCA-1, and FHIT. The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The nucleic acids corresponding to promoters of TSGs isolated from the subject selected from the group consisting of DAPK, RAR-beta, p14, p73, MGMT, APC, SOCS-1, BRCA-1, and FHIT can be any nucleic acids where it is desirable to detect the presence of a differentially methylated CpG island. The CpG island is a CpG rich region of a nucleic acid sequence. The nucleic acids can correspond, for example, to a sequence encoding the promoter of TSGs, selected from the group consisting of DAPK, RAR-beta, p14, p73, MGMT, APC, SOCS-1, BRCA-1, and FHIT.

Any nucleic acid sample, in purified or nonpurified form, can be utilized in accordance with the present invention, provided it contains, or is suspected of containing, a nucleic acid sequence containing a target locus (e.g., CpG-containing nucleic acid. One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G:C base pairs. In certain regions, the density of CpG doublets reaches the predicted value; it is increased by ten fold relative to the rest of the genome. CpG islands have an average G:C content of about 60%, compared with the 40% average in bulk DNA. The islands take the form of stretches of DNA typically about one to two kilobases long. There are about 45,000 such islands in the human genome.

In many genes, the CpG islands begin just upstream of a promoter and extend downstream into the transcribed region. Methylation of a CpG island at a promoter usually prevents expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

In general, the CpG-containing nucleic acid is DNA. However, invention methods may employ, for example, samples that contain DNA, or DNA and RNA, including messenger RNA, wherein DNA or RNA may be single stranded or double stranded, or a DNA-RNA hybrid may be included in the sample. A mixture of nucleic acids may also be employed. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. The nucleic acid-containing sample used for determination of the state of methylation of nucleic acids contained in the sample or detection of methylated CpG islands may be extracted by a variety of techniques such as that described by Sambrook, et al. (Molecular Cloning: A Laboratory Manual, Cold Spring Harbor, N.Y., 1989; incorporated in its entirety herein by reference).

A nucleic acid can contain a regulatory region, which is a region of DNA that encodes information that directs or controls transcription of the nucleic acid. Regulatory regions include at least one promoter. Promoters may be located in the 5′ or 3′ regions of the gene. Promoter regions, in whole or in part, of a number of nucleic acids can be examined for sites of CG-island methylation.

Nucleic acids isolated from a subject are obtained in a biological specimen from the subject. The nucleic acid may be isolated from tissue or a biological fluid, such as blood, plasma serum, and urine. Tissue, blood, plama serum, and urine are obtained by various procedures known to those of skill in the art.

In one aspect of the invention, the state of methylation the sample nucleic acids comprising promoters of tumor suppressor genes selected from the group consisting of DAPK, RAR-beta, p14, p73, MGMT, APC, SOCS-1, BRCA-1, and FHIT obtained from a subject is compared with the same regions of the nucleic acid in a subject not having bladder urethelial carcinoma. Nucleic acids from a subject not having a bladder urethelial carcinoma contain no detectable methylated alleles when the same nucleic acids are examined.

A method for determining the methylation state of nucleic acids is described in U.S. Pat. No. 6,017,704 which is incorporated herein in its entirety and described briefly herein. Determining the methylation state of the nucleic acid includes amplifying the nucleic acid by means of oligonucleotide primers that distinguishes between methylated and unmethylated nucleic acids.

Two or more markers corresponding to promoters of tumor suppressor genes selected from the group consisting of DAPK, RAR-beta, p14, p73, MGMT, APC, SOCS-1, BRCA-1, and FHIT can also be screened simultaneously in a single amplification reaction to generate a low cost, reliable screening test for bladder urethelial carcinomas. A combination of DNA markers for CpG-rich regions of nucleic acid may be amplified in a single amplification reaction. The markers are multiplexed in a single amplification reaction, for example, by combining primers for more than one locus. For example, DNA from a urine sample can be amplified with three or more different unlabeled or randomly labeled primer sets in the same amplification reaction. Especially useful are three or more markers comprising DNA of promoters of TSGs selected form group consisting of DAPK, RAR-beta, p14, p73, MGMT, APC, SOCS-1, BRCA-1, and FHIT. The reaction products are separated on a denaturing -polyacrylamide gel, for example, and then exposed to film or stained with ethidium bromide for visualization and analysis. By analyzing a panel of markers, there is a greater probability of producing a more useful methylation profile for a subject.

For example, a screening technique, referred to herein as “multiplex methylation-specific PCR” is a unique version of methylation-specific PCR. Methylation-specific PCR is described in U.S. Pat. Nos. 5,786,146, 6,200,756, 6,017,704 and 6,265,171, each of which is incorporated herein by reference in its entirety. Multiplex methylation-specific PCR utilizes MSP primers for a multiplicity of markers, in a two-stage nested PCR amplification reaction. The primers used in the first PCR reaction are selected to amplify a larger portion of the target sequence than the primers of the second PCR reaction. The primers used in the first PCR reaction are referred to herein as “external primers” or DNA primers” and the primers used in the second PCR reaction are referred to herein as “MSP primers.” Two sets of primers (i.e., methylated and unmethylated for each of the markers targeted in the reaction) are used as the MSP primers.

If the sample is impure (e.g., plasma, serum, urine), it may be treated before amplification with a reagent effective for lysing the cells contained in the tissues or biological fluids of the sample, and for exposing the nucleic acid(s) contained therein. Methods for purifying or partially purifying nucleic acid from a sample are well known in the art (e.g., Sambrook et al., Molecular Cloning: A Laboratory Manual, Cold Spring Harbor Press, 1989, herein incorporated by reference).

Detection of differential methylation can be accomplished by contacting a nucleic acid sample with a methylation sensitive restriction endonuclease that cleaves only unmethylated CpG sites under conditions and for a time to allow cleavage of unmethylated nucleic acid. The sample is further contacted with an isoschizomer of the methylation sensitive restriction endonuclease that cleaves both methylated and unmethylated CpG-sites under conditions and for a time to allow cleavage of methylated nucleic acid. Oligonucleotides are added to the nucleic acid sample under conditions and for a time to allow ligation of the oligonucleotides to nucleic acid cleaved by the restriction endonuclease, and the digested nucleic acid is amplified by conventional methods, such as PCR wherein primers complementary to the oligonucleotides are employed. Following identification, the methylated CpG-containing nucleic acid can be cloned, using methods well known to those of skill in the art (see Sambrook et al., Molecular Cloning: A Laboratory Manual, Cold Spring Harbor Press, 1989).

As used herein, a “methylation sensitive restriction endonuclease” is a restriction endonuclease that includes CG as part of its recognition site and has altered activity when the C is methylated as compared to when the C is not methylated. Preferably, the methylation sensitive restriction endonuclease has inhibited activity when the C is methylated (e.g., Sma l). Specific non-limiting examples of methylation sensitive restriction endonucleases include Sma I, BssHII, or HpaII, MspI, BSTUI, and NotI. Such enzymes can be used alone or in combination. Other methylation sensitive restriction endonucleases will be known to those of skill in the art and include, but are not limited to SacII, and EagI, for example. An “isoschizomer” of a methylation sensitive restriction endonuclease is a restriction endonuclease that recognizes the same recognition site as a methylation sensitive restriction endonuclease but cleaves both methylated and unmethylated CGs. Those of skill in the art can readily determine appropriate conditions for a restriction endonuclease to cleave a nucleic acid (see Sambrook et al., Molecular Cloning: A Laboratory Manual, Cold Spring Harbor Press, 1989).

A nucleic acid of interest is cleaved with a methylation sensitive endonuclease. Cleavage with the methylation sensitive endonuclease creates a sufficient overhang on the nucleic acid of interest, i.e., sufficient to allow specific hybridization of an oligonucleotide of interest. Following cleavage with the isoschizomer, the cleavage product can still have a sufficient overhang. An “overhang” refers to nucleic acid having two strands wherein the strands end in such a manner that a few bases of one strand are not base paired to the other strand. A “sufficient overhang” refers to an overhang of at least two bases in length or four or more bases in length. An overhang of a specific sequence on the nucleic acid of interest may be desired in order for an oligonucleotide of interest to hybridize. In this case, the isoschizomer can be used to create the overhang having the desired sequence on the nucleic acid of interest.

Cleavage with a methylation sensitive endonuclease results in a reaction product of the nucleic acid of interest that has a blunt end or an insufficient overhang. “Blunt end” refers to a flush ending of two stands, the sense stand and the antisense strand, of a nucleic acid. Once a sufficient overhang is created on the nucleic acid of interest, an oligonucleotide is ligated to the nucleic acid of interest, which has been cleaved by the methylation specific restriction endonuclease. “Ligation” is the attachment of two nucleic acid sequences by base pairing of substantially complementary sequences and/or by the formation of covalent bonds between two nucleic acid sequences.

An adaptor can be utilized to create DNA ends of desired sequence and overhang. An “adaptor” is a double-stranded nucleic acid sequence with one end that has a sufficient single-stranded overhang at one or both ends such that the adaptor can be ligated by base-pairing to a sufficient overhang on a nucleic acid of interest that has been cleaved by a methylation sensitive restriction enzyme or an isoschizomer of a methylation sensitive restriction enzyme. Adaptors can be obtained commercially. Alternatively, two oligonucleotides that are substantially complementary over their entire sequence except for the region(s) at the 5′ and/or 3′ ends that will form a single stranded overhang can be used to form an adaptor. The single stranded overhang on the adapter is selected to be complementary to an overhang on the nucleic acid cleaved by a methylation sensitive restriction enzyme or an isoschizomer of a methylation sensitive restriction enzyme, such that the overhang on the nucleic acid of interest will base pair with the 3′ or 5′ single stranded end of the adaptor under appropriate conditions. The conditions will vary depending on the sequence composition (GC vs AT), the length, and the type of nucleic acid (see Sambrook et al., Molecular Cloning: A Laboratory Manual, 2nd Ed.; Cold Spring Harbor Laboratory Press, Plainview, N.Y., 1998).

Following the ligation of the oligonucleotide to the nucleic acid of interest, the nucleic acid of interest is amplified using a primer complementary to the oligonucleotide. Environmental conditions conducive to synthesis include the presence of nucleoside triphosphates, an agent for polymerization, such as DNA polymerase, and suitable temperature and pH. The primer is preferably single stranded for maximum efficiency in amplification, but may be double stranded. If double stranded, the primer is first treated to separate its strands before being used to prepare extension products. The primer can be an oligodeoxyribonucleotide. The primer must be sufficiently long to prime the synthesis of extension products in the presence of the agent for polymerization. The exact length of the primer will depend on many factors, including temperature, buffer composition (i.e., salt concentration), and nucleotide composition. The oligonucleotide primer typically contains 12-20 or more nucleotides, although it may contain fewer nucleotides.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions that allow the agent for polymerization to perform. In other words, the primers should have sufficient complementarity with a 5′ and 3′ oligonucleotide to hybridize therewith and permit amplification of CpG containing nucleic acid sequence.

Primers of the invention are employed in the amplification process, which is an enzymatic chain reaction that produces exponentially increasing quantities of target locus relative to the number of reaction steps involved (e.g., polymerase chain reaction or PCR). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

The oligonucleotide primers used in invention methods may be prepared using any suitable method, such as conventional phosphotriester and phosphodiester methods or automated embodiments thereof. In one such automated embodiment, diethylphos-phoramidites are used as starting materials and may be synthesized as described by Beaucage, et al. (Tetrahedron Letters, 22:1859-1862, 1981). One method for synthesizing oligonucleotides on a modified solid support is described in U.S. Pat. No. 4,458,066.

Another method for detecting a methylated CpG-containing nucleic acid includes contacting a nucleic acid-containing specimen with an agent that modifies unmethylated cytosine, amplifying the CpG-containing nucleic acid in the specimen by means of CpG-specific oligonucleotide primers, wherein the oligonucleotide primers distinguish between modified methylated and non-methylated nucleic acid and detecting the methylated nucleic acid. The amplification step is optional and although desirable, is not essential. The method relies on the PCR reaction itself to distinguish between modified (e.g., chemically modified) methylated and unmethylated DNA.

The term “modifies” as used herein means the conversion of an unmethylated cytosine to another nucleotide that will facilitate methods to distinguish the unmethylated from the methylated cytosine. The agent can modify unmethylated cytosine to uracil. For example, the agent used for modifying unmethylated cytosine can be sodium bisulfite; however, other agents that similarly modify unmethylated cytosine, but not methylated cytosine, can also be used in the method. Sodium bisulfite (NaHSO₃) reacts readily with the 5,6-double bond of cytosine, but poorly with methylated cytosine. Cytosine reacts with the bisulfite ion to form a sulfonated cytosine reaction intermediate that is susceptible to deamination, giving rise to a sulfonated uracil. The sulfonate group can be removed under alkaline conditions, resulting in the formation of uracil. Uracil is recognized as a thymine by Taq polymerase. Therefore after PCR, the resultant product contains cytosine only at the position where 5-methylcytosine occurs in the starting template DNA.

The primers used in the invention for amplification of the CpG-containing nucleic acid in the specimen, after bisulfite modification, specifically distinguish between untreated or unmodified DNA, methylated, and non-methylated DNA. MSP primers for the non-methylated DNA preferably have a T in the 3′ CG pair to distinguish it from the C retained in methylated DNA, and the complement is designed for the antisense primer. MSP primers usually contain relatively few Cs or Gs in the sequence since the Cs will be absent in the sense primer and the Gs absent in the antisense primer (C becomes modified to U (uracil) which is amplified as T (thymidine) in the amplification product).

The primers of the invention embrace oligonucleotides of sufficient length and appropriate sequence so as to provide specific initiation of polymerization on a significant number of nucleic acids in the polymorphic locus. Where the nucleic acid sequence of interest contains two strands, it is necessary to separate the strands of the nucleic acid before it can be used as a template for the amplification process. Strand separation can be effected either as a separate step or simultaneously with the synthesis of the primer extension products. This strand separation can be accomplished using various suitable denaturing conditions, including physical, chemical, or enzymatic means, the word “denaturing” includes all such means. One physical method of separating nucleic acid strands involves heating the nucleic acid until it is denatured. Typical heat denaturation may involve temperatures ranging from about 80° C. to about 105° C. for times ranging from about 1 to about 10 minutes. Strand separation may also be induced by an enzyme from the class of enzymes known as helicases or by the enzyme RecA, which has helicase activity, and in the presence of riboATP, is known to denature DNA. The reaction conditions suitable for strand separation of nucleic acids with helicases are described by Kuhn Hoffmann-Berling (CSH-Quantitative Biology, 43:63, 1978) and techniques for using RecA are reviewed in C. Radding (Ann. Rev. Genetics, 16:405-437, 1982).

When complementary strands of nucleic acids are separated, regardless of whether the nucleic acid was originally double or single stranded, the separated strands are ready to be used as a template for the synthesis of additional nucleic acid strands. This synthesis is performed under conditions allowing hybridization of primers to templates to occur. Generally synthesis occurs in a buffered aqueous solution, generally at a pH of about 7 to about 9. Preferably, a molar excess (for genomic nucleic acid, usually about 108:1 primer:template) of the two oligonucleotide primers is added to the buffer containing the separated template strands. It is understood, however, that the amount of complementary strand may not be known if the process of the invention is used for diagnostic applications, so that the amount of primer relative to the amount of complementary strand cannot be determined with certainty. As a practical matter, however, the amount of primer added will generally be in molar excess over the amount of complementary strand (template) when the sequence to be amplified is contained in a mixture of complicated long-chain nucleic acid strands. Large molar excess is preferred to improve the efficiency of the process.

The deoxyribonucleoside triphosphates dATP, dCTP, dGTP, and dTTP are added to the synthesis mixture, either separately or together with the primers, in adequate amounts and the resulting solution is heated to about 90° C. to about 100° C. from about 1 to about 10 minutes, preferably from 1 to 4 minutes. After this heating period, the solution is allowed to cool to approximately room temperature, which is preferable for the primer hybridization. To the cooled mixture is added an appropriate agent for effecting the primer extension reaction (called herein “agent for polymerization”), and the reaction is allowed to occur under conditions known in the art. The agent for polymerization may also be added together with the other reagents if it is heat stable. This synthesis (or amplification) reaction may occur at room temperature up to a temperature above which the agent for polymerization no longer functions. Thus, for example, if DNA polymerase is used as the agent, the temperature is generally no greater than about 40.degree. C. Most conveniently the reaction occurs at room temperature.

The agent for polymerization may be any compound or system that will function to accomplish the synthesis of primer extension products, including enzymes. Suitable enzymes for this purpose include, for example, E. coli DNA polymerase I, Klenow fragment of E. coli DNA polymerase I, T4 DNA polymerase, other available DNA polymerases, polymerase muteins, reverse transcriptase, and other enzymes, including heat-stable enzymes (i.e., those enzymes which perform primer extension after being subjected to temperatures sufficiently elevated to cause denaturation such as Taq DNA polymerase, and the like). Suitable enzymes will facilitate combination of the nucleotides in the proper manner to form the primer extension products that are complementary to each locus nucleic acid strand. Generally, the synthesis will be initiated at the 3′ end of each primer and proceed in the 5′ direction along the template strand, until synthesis terminates, producing molecules of different lengths. There may be agents for polymerization, however, which initiate synthesis at the 5′ end and proceed in the other direction, using the same process as described above.

The method of amplifying can be by PCR, as described herein and as is commonly used by those of ordinary skill in the art. However, alternative methods of amplification have been described and can also be employed. PCR techniques and many variations of PCR are known. Basic PCR techniques are described by Saiki et al. (1988 Science 239:487-491) and by U.S. Pat. Nos. 4,683,195, 4,683,202 and 4,800,159, each of which is incorporated herein by reference.

The conditions generally required for PCR include temperature, salt, cation, pH and related conditions needed for efficient copying of the master-cut fragment. PCR conditions include repeated cycles of heat denaturation (i.e., heating to at least about 95° C.) and incubation at a temperature permitting primer: adaptor hybridization and copying of the master-cut DNA fragment by the amplification enzyme. Heat stable amplification enzymes like the pwo, Thermus aquaticus or Thermococcus litoralis DNA polymerases, which eliminate the need to add enzyme after each denaturation cycle, are commercially available. The salt, cation, pH and related factors needed for enzymatic amplification activity are available from commercial manufacturers of amplification enzymes.

As provided herein an amplification enzyme is any enzyme which can be used for in vitro nucleic acid amplification, e.g. by the above-described procedures. Such amplification enzymes include pwo, Escherichia coli DNA polymerase I, Klenow fragment of E. coli DNA polymerase I, T4 DNA polymerase, T7 DNA polymerase, Thermus aquaticus (Taq) DNA polymerase, Thermococcus litoralis DNA polymerase, SP6 RNA polymerase, T7 RNA polymerase, T3 RNA polymerase, T4 polynucleotide kinase, Avian Myeloblastosis Virus reverse transcriptase, Moloney Murine Leukemia Virus reverse transcriptase, T4 DNA ligase, E. coli DNA ligase or Q_{β} replicase. Preferred amplification enzymes are the pwo and Taq polymerases.

Once amplified, the nucleic acid can be attached to a solid support, such as a membrane, and can be hybridized with any probe of interest, to detect any nucleic acid sequence. Several membranes are known to one of skill in the art for the adhesion of nucleic acid sequences. Specific non-limiting examples of these membranes include nitrocellulose (NITROPURE) or other membranes used in for detection of gene expression such as polyvinylchloride, diazotized paper and other commercially available membranes such as GENESCREEN, ZETAPROBE (Biorad), and NYTRAN. Methods for attaching nucleic acids to these membranes are well known to one of skill in the art. Alternatively, screening can be done in a liquid phase.

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

The probe of interest can be detectably labeled, for example, with a radioisotope, a fluorescent compound, a bioluminescent compound, a chemiluminescent compound, a met al chelator, or an enzyme. Those of ordinary skill in the art will know of other suitable labels for binding to the probe, or will be able to ascertain such, using routine experimentation.

Another embodiment of the invention provides a method of determining a predisposition to a bladder urethelial cell carcinoma in a subject comprising determining the state of methylation of three or more nucleic acids isolated from the subject, wherein the nucleic acid are promoters of tumor suppressor genes selected from the group consisting of DAPK, RAR-beta, p14, p73, MGMT, APC, SOCS-1, BRCA-1, and FHIT; and wherein the state of methylation of one or more nucleic acids as compared with the state of methylation of said nucleic acid from a subject not having a predisposition to bladder urethelial cell carcinoma is indicative of a bladder urethelial cell carcinoma in the subject.

Another embodiment of the invention provides a method for diagnosing bladder urethelial cell carcinoma in a subject comprising contacting a nucleic acid-containing specimen from the subject with agents that provide a determination of the methylation state of nucleic acids in the specimen. The nucleic acids correspond to promoters of tumor suppressor genes selected from the group consisting of DAPK, RAR-beta, p14, p73, MGMT, APC, SOCS-1, BRCA-1, and FHIT. The methylation state of at least one region of the nucleic acids comprising promoters of tumor suppressor genes selected from the group consisting of DAPK, RAR-beta, p14, p73, MGMT, APC, SOCS-1, BRCA-1, and FHIT is then identified. The methylation state of at least one region of the nucleic acids that is different from the methylation state of the same region of the same nucleic acid in a subject not having the bladder urethelial carcinoma is indicative of bladder urethelial carcinoma in the subject.

Methods in accordance with the present invention are ideally suited for the preparation of a kit. Therefore, in accordance with another aspect of the present invention, there is provided a kit that can be used for the detection of a bladder urethelial carcinoma in a subject. Invention kits include a carrier means compartmentalized to receive a sample therein, one or more containers comprising a first container containing a reagent which modifies unmethylated cytosine and a second container containing primers for amplification of a CpG-containing nucleic acid, wherein the primers distinguish between modified methylated and nonmethylated nucleic acid.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing an oligonucleotide for ligation to nucleic acid cleaved by a methylation sensitive restriction endonuclease. One or more container means can also be included comprising a primer complementary to the oligonucleotide. In addition, one or more container means can also be included which comprise a methylation sensitive restriction endonuclease. One or more container means can also be included containing an isoschizomer of said methylation sensitive restriction enzyme.

### EXAMPLE

To facilitate accurate detection of bladder transitional cell carcinoma in urine, we have tested the usefulness of promoter methylation profiling of 14 tumor suppressor genes in 55 cases of voided and instrumented urine samples from patients with known clinical outcomes. Fifty-five cased including 30 papillary TCC (15 low-grade and 15 high-grade), 10 flat CIS and 15 benign/reactive bladder urothelium. DNA promoter methylation profiling of 14 tumor suppressor genes, APC, RAR-beta, p14, p15, p16, p73, RASSF1a, hMLH1, DAPK, MGMT, APC, SOCS-1, BRCA-1 and FHIT, was analyzed using nested multiplex methylation specific PCR.

Methylation of a panel of TSG promoters can be used to detect bladder TCC. The CpG islands/TSGs that are frequently methylated in bladder TCC, but not in benign/reactive urothelial cells, are DAPK, RAR-beta, p14, p73, MGMT, APC, SOCS-1, BRCA-1, and FHIT. Concurrent promoter methylation of 3 or more CpG islands distinguishes papillary TCC and flat CIS from benign/reactive urethelial cells. Using CMMG promoter methylation profiling, the sensitivity of detecting bladder TCC in urine is about 87% with a specificity approaching 100%. In comparison, the sensitivity of cyctological detection of low-grade papillary TCC, high-grade papillary TCC and flat CIS are about 13%, about 53%, and about 80% respectively, About 85% of cytologically misdiagnosed cases as either “negative” or “atypical” can be re-classified as “positive for TCC” by applying CMMG methylation profiling of the panel of 15 TSG. Methylation of TSG promoters was not seen in 5 cases of BK (polyoma) virus-infected urine, while false positive FISH results have been reported in BK (polyoma) virus-infected urothelium.

Although the invention has been described with reference to the presently preferred embodiment, is should be understood that various modifications can be made without departing from the spirit of the invention. Accordingly, the invention is limited only by the following claims.

1. A diagnostic method for bladder urethelial carcinoma, the method comprising:
obtaining an isolated nucleotide sample from a subject; and
detecting the promoter methylation of at least three tumor suppressor genes selected form group consisting of DAPK, RAR-beta, p14, p73, MGMT, APC, SOCS-1, BRCA-1, and FHIT.

2. The method of claim 1, the isolated nucleic sample comprising isolated genomic DNA.

3. The method of claim 2, the isolated genomic DNA being obtained from at least one of tissue or biological fluid of the subject.

4. The method of claim 1, the methylation detection of the at least three promoters of tumor suppressor genes distinguishing bladder transition cell carcinoma from benign or malignant urethelial cells.

5. The method of claim 2, the methylation detection of the at three promoters of tumor suppressor genes being performed by multiplex methylation specific PCR.

6. The method of claim 1, the isolated nucleotide material being obtained from at least one of voided or instrumented urine samples.

7. The method of claim 1, the methylation detection measuring the methylation of CpG islands of the promoters of the tumor suppressor genes.

8. The method of claim 5, the methylation detection of the at least three promoters of tumor suppressor genes distinguishing papillary transition cell carcinoma and flat urethelial cell carcinoma from benign or malignant urethelial cells.

9. A diagnostic method for distinguishing flat and papillary bladder transition cell carcinoma from benign or malignant urethelial cells in a subject, the method comprising:
obtaining an isolated nucleotide sample from the subject; and
detecting the promoter methylation of at least three tumor suppressor genes selected form group consisting of DAPK, RAR-beta, p14, p73, MGMT, APC, SOCS-1, BRCA-1, and FHIT.

10. The method of claim 9, the isolated nucleic sample comprising isolated genomic DNA.

11. The method of claim 10, the isolated genomic DNA being obtained from at least one of tissue or biological fluid of the subject.

12. The method of claim 2, the methylation detection of the at three promoters of tumor suppressor genes being performed by multiplex methylation specific PCR.

13. The method of claim 1, the isolated nucleotide material being obtained from at least one of voided or instrumented urine samples.

14. The method of claim 9, the methylation detection measuring the methylation of CpG islands of the promoters of the tumor suppressor genes.

15. A diagnostic method for determining a subject's predisposition to bladder urethelial carcinoma, the method comprising:
obtaining an isolated nucleotide sample from a subject; and
detecting the promoter methylation of at least three tumor suppressor genes selected form group consisting of DAPK, RAR-beta, p14, p73, MGMT, APC, SOCS-1, BRCA-1, and FHIT.

16. The method of claim 15, the isolated nucleic sample comprising isolated genomic DNA.

17. The method of claim 16, the isolated genomic DNA being obtained from at least one of tissue or biological fluid of the subject.

18. The method of claim 15, the methylation detection of the at least three promoters of tumor suppressor genes distinguishing bladder transition cell carcinoma from benign or malignant urethelial cells.

19. The method of claim 16, the methylation detection of the at three promoters of tumor suppressor genes being performed by multiplex methylation specific PCR.

20. The method of claim 15, the isolated nucleotide material being obtained from at least one of voided or instrumented urine samples.

21. The method of claim 1, the methylation detection measuring the methylation of CpG islands of the promoters of the tumor suppressor genes.

22. The method of claim 15, the methylation detection of the at least three promoters of tumor suppressor genes distinguishing papillary transition cell carcinoma and flat urethelial cell carcinoma from benign or malignant urethelial cells.
